(19) United States
(12) Patent Application Publication
(10) Pub. No.: US 2022/0184558 A1
(43) Pub. Date: Jun. 16, 2022

(54) FILTRATION APPARATUS

(71) Applicant: CELL THERAPY CATAPULT LIMITED, London (GB)

(72) Inventors: Daria POPOVA, London (GB); Nicholas Richard GADDUM, London (GB); Mark Anthony SITCOSKE, Smithfield, RI (US); Michael Matthew GIULIANO, Smithfield, RI (US)

(21) Appl. No.: 17/432,247

(22) PCT Filed: Feb. 24, 2020
(86) PCT No.: PCT/GB2020/050437

§ 371 Date: Aug. 19, 2021

(30) Foreign Application Priority Data
Mar. 1, 2019 (GB) 1902816.6

(51) Int. Cl. B01D 65/02; B01D 61/08; B01D 61/12; B01D 61/18; B01D 61/22; C12M 1/00

(52) U.S. Cl. CPC B01D 65/02; B01D 61/08; B01D 61/12; B01D 61/18; B01D 2321/185; C12M 47/02; B01D 2311/25; B01D 2315/10; B01D 2313/243; B01D 61/22

(57) ABSTRACT

A multiple-loop tangential flow filtration apparatus for concentrating fluids is described herein. The apparatus comprises a plurality of tube loops for receiving fluid therethrough, each tube loop comprising a respective filter, and a common feed pump for driving the fluid across each respective filter. The plurality of tube loops are coupled to the common pump via a common feed line.

### FIELD OF THE INVENTION

The present disclosure relates to a filtration apparatus, and in particular a tangential filtration apparatus such as a tangential flow filtration apparatus.

### BACKGROUND

The expansion of cells in large volumes is a necessary step for many cell therapies, to allow the generation of sufficient product to meet patient demand. Particularly, allogeneic cell therapies require the culture and expansion of cells from a single individual, for ultimate administration to multiple patients, necessitating large scale cell expansion. There exists a requirement to concentrate cells after expansion into an appropriate volume for downstream applications and for patient administration.

Tangential flow filtration (TFF) is an example of tangential filtration and is a well-known technique which has been disclosed in the art as having many different applications, including a well published application in cell concentration. TFF provides a continuous process, in which a sample (e.g. cells in culture medium) travels tangentially across a filter or membrane (i.e. in parallel) rather than into a filter (e.g. in a perpendicular direction), where the resulting fluid shearing stress helps reduce the blockage or fouling of the membrane with material. TFF is often advantageous over traditional filtration methods where sample is passed directly through a membrane in a perpendicular direction, ultimately causing filter blockage and resulting in a dead end process.

Typically, TFF systems of the art are based on a single tube loop system, where sample is passed around the system and across a membrane to remove filtrate one or more times, thus concentrating the sample. In general, industry standard systems typically result in a 10× concentration of samples. Although some systems claim a 70× concentration of samples, this is very difficult, if not impossible to achieve in practice. If a higher concentration of sample is desired, it is currently necessary to employ multiple single loop TFF systems, with decreasing tube loop diameters, which is cumbersome, time consuming, is problematic in terms of sterility and represents an expensive way to concentrate to appropriate levels. Therefore, there is a need for the development of an improved single TFF system which is capable of achieving higher levels of sample concentration without the need to employ multiple systems.

Another example of tangential filtration is alternating tangential filtration (ATF). ATF typically involves the use of an alternating flow pump such as a diaphragm pump that repeatedly reverses the flow of fluid across a filter membrane to reduce the possibility of fouling of the filter membrane.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

The present inventors have developed a multiple loop TFF system which is capable of achieving 168× concentration of samples, where the system currently comprises two tube loops (typically a large loop and a small loop) and a single sample pump and pump head. The inventors have shown that a 10 L sample can be reduced to 50-60 ml using the newly developed system, therefore negating the need to employ multiple TFF systems. The level of concentration achieved with the newly developed system is extremely advantageous over the prior art systems and should address an industry need.

The system typically employs one tube of a higher capacity (for example a larger diameter (in the large loop) capable of processing or handling a higher flow rate and/or volume of fluid) and one of a lower capacity (for example a smaller diameter (in the small loop) capable of processing or handling a lower flow rate and/or volume of fluid), where the sample is firstly passed through the large tube loop and is subsequently moved to and passed through the smaller tube loop, allowing the high concentration of the sample. The membranes/filters used in the two loops are distinct, where the large loop employs more permeable membrane tubes in parallel to increase the effective filter surface. It will be understood that a loop being described as higher capacity as compared to a loop having a lower capacity may (i) have a greater volume (e.g. a volume which is at least 10, 20, 30, 40, 50, 60, 70, 80, 90 or 95% greater) and/or (ii) be able to remove fluid from the loop at a greater rate than the lower capacity loop (e.g. at a rate which is at least 10, 20, 30, 40, 50, 60, 70, 80, 90 or 95% greater)—for example, the higher capacity loop may comprise a filter membrane with a larger surface area (which may therefore have more fibres). Various modifications have been made to the system, to allow the use of tube loops of differing diameters, for example, the use of a single common pump (for example, a single common pump head) operating on a common feed line, and optionally the introduction of air inlets. It will of course be understood that although a larger and a smaller tube loop may be used, at various points the tube loops may have dimensions in common—for example the loops may share the same sized common feed line, and/or each tube loop may comprise tapered portion(s) so as to couple with the same feature, such as to a selector.

Accordingly in a first aspect of the disclosure there is provided a multiple-loop tangential flow filtration apparatus for concentrating fluids. The apparatus comprises a plurality of tube loops for receiving fluid therethrough, each tube loop comprising a respective filter, and a common feed pump for driving the fluid across each respective filter. Each respective filter is configured for use with tangential flow filtration. The plurality of tube loops are coupled to the common feed pump via a common feed line. The common feed pump is preferably a self-priming pump, such as a diaphragm or gear pump.

The multiple-loop tangential flow filtration apparatus in example embodiments comprises a first tube loop comprising a corresponding first feed vessel, and a second tube loop comprising a corresponding second feed vessel. An input of each tube loop may be coupled to the common feed line, and an output of the first tube loop may be coupled to an input of the first feed vessel. An output of the second tube loop may be coupled to an input of the second feed vessel such that the retentate from the first tube loop is returnable to the first feed vessel and the retentate from the second tube loop is returnable to the second feed vessel.

In some examples where there are more tube loops, each respective tube loop comprises a corresponding feed vessel, and wherein an input of each tube loop is coupled to the common feed line, and an output of each tube loop is coupled to an input of the respective feed vessel such that the retentate from each respective tube loop is returnable to a corresponding feed vessel.

The common feed line may comprise a selector such as a switch (for example, a solenoid valve and/or tube clamp) for selecting which feed vessel from which to draw fluid into the common feed line and additionally or alternatively a selector such as a switch (for example, a solenoid valve and/or tube clamp) for selecting which filter of each tube loop to direct the fluid from the common feed line towards. It will be understood that a selector may comprise a plurality of solenoid valves and/or tube clamps that function together to provide the functionality of a selector for selecting which line fluid is fed into and/or drawn from. For example, the selector may comprise a Y connection with a means such as a clamp or pinch valve coupled to each branch of the Y connection operable to close a corresponding tube or line downstream of the Y connection to select which of the two lines coupled to respective branches of the Y connection fluid is directed along.

Preferably each of the plurality of tube loops has a different internal cross-sectional area (for example a different internal diameter if the tubes are circular), and tube loops with a smaller internal cross-sectional area have a shorter total loop path length than tube loops with a larger internal cross-sectional area. In this way the apparatus can be arranged to progressively reduce the dead volume in the system to ensure that the amount of retentate (which can be very expensive when handling biological samples) left in the apparatus can be reduced to a minimum.

In some examples the first or each feed vessel has a first air inlet for allowing the corresponding feed vessel to drain completely, and in some examples the first or each feed vessel has a tapered portion proximal to the outlet coupled to the common feed line.

In some examples the apparatus may be arranged to limit or inhibit the occurrence of a stream of fluid dripping into a static body of fluid (which may produce bubbles and/or damage the biological sample in the fluid). For example, the output of each tube loop may be coupled to the input of each respective feed vessel with a line that extends through the feed vessel from the input and down proximal to an outlet that is coupled to the common feed line, such that fluid that returns to the feed vessel returns near to the bottom of the feed vessel.

In some examples the first tube loop comprises a second air inlet for allowing at least a portion of the first tube loop to drain completely. In other examples each tube loop comprises a second air inlet for allowing at least a portion of the first tube loop to drain completely. For example, the second air inlet may be configured to allow a feed line coupling the common feed line to the first (or a respective) feed vessel to drain completely, and in some examples may allow fluid in the feed line, pump, filter and return line (returning to the feed vessel) to be drained.

Each tube loop may be coupled to a common waste vessel via a common waste line. The common waste line may be coupled to a common filtrate pump for removing the filtrate from each filter. In some examples the common waste line comprises a selector such as a switch for selecting which filter filtrate is received from.

In some examples the apparatus comprises a controller for controlling the flow of fluid through the apparatus. The controller may be coupled (for example with a physical wired connection and/or wirelessly) to the selectors and/or the pumps. The controller is configured to receive information relating to the mass of fluid in the apparatus, determine which tube loop to direct fluid through based on the received information relating to the mass of fluid, and direct the fluid through a corresponding tube loop based on the determination. The controller may further be configured to receive information relating to pressure, such as the transmembrane pressure of fluid across each filter of each tube loop in the apparatus, and control operation of the common filtrate pump based on the received pressure information. In such examples the controller may be configured to maintain the transmembrane pressure across a filter or each filter in a selected range to inhibit blockage of the filter. The controller may be configured to control operation of the common feed pump and/or common waste pump and/or selectors to control the fluid flow through the apparatus, for example to maintain the transmembrane pressure. The controller may be configured to do this by sending control signals (either via a wired or wireless connection) to the common feed pump and/or common waste pump and/or selectors to control their operation. In some examples the controller may also be configured to receive signals from the common feed pump and/or common waste pump and/or selectors (as well as from sensors such as mass and/or pressure sensors as described in more detail below) to determine if the apparatus is functioning correctly and to determine if there is an error state anywhere in the apparatus.

The apparatus may further comprise means for identifying the mass of fluid in at least the first feed vessel (and optionally in each feed vessel), and the controller may be configured to direct the fluid from through the first tube loop to through the second tube loop in response to the mass of fluid in the first feed vessel falling below a selected threshold. The selected threshold may be selected based on the volume of the feed vessel, for example the selected threshold may be selected to correspond to 10%, for example 5%, of the volume of the feed vessel.

In another aspect of the disclosure there is provided a multiple-loop tangential flow filtration apparatus for concentrating fluids comprising a first tube loop comprising a corresponding first feed vessel and a first filter and a second tube loop comprising a corresponding second feed vessel and a second filter. The apparatus also comprises a common pump for driving the fluid across each respective filter of each tube loop, wherein the first and second tube loops are coupled to the common pump via a common feed line, the common feed line comprising a first selector for selecting which feed vessel from which to draw fluid into the common feed line and a second selector to select which filter of each tube loop to direct the fluid from the common feed line towards. The apparatus also comprises a controller for controlling the flow of fluid through the apparatus. The controller is configured to receive information relating to the mass of fluid in at least one of the feed vessels, determine which tube loop to direct fluid through based on the received information relating to the mass of fluid in at least one of the feed vessels, and direct the fluid through a corresponding tube loop based on the determination by controlling operation of the first and second selectors.

In some examples the controller is configured to direct fluid through the second tube loop in response to the mass of fluid in the first feed vessel falling below a selected threshold. The selected threshold may be selected based on the volume of the feed vessel, for example the selected threshold may be selected to correspond to 10%, for example 5%, of the volume of the feed vessel.

In some examples the first feed vessel has a first air inlet for allowing the first feed vessel to drain completely and the first tube loop comprises a second air inlet for allowing at least a portion of the first tube loop to drain completely. In such examples directing fluid to flow through the second tube loop comprises operating the second selector to direct fluid from the common feed line the second tube loop, and opening the first air inlet to allow air to flow into the first feed vessel to drain the first feed vessel. In response to determining that the mass of fluid in the first feed vessel has fallen below a second selected threshold, the controller is configured to open the second air inlet to allow air to flow into a feed line coupling the common feed line to the first feed vessel, receive an indication that the fluid has drained from the feed line coupling the common feed line to the first feed vessel and, in response, operate the first selector coupled to the common feed line so that the common feed line receives fluid from the second feed vessel instead of the first feed vessel.

In some examples receiving an indication that the fluid has drained from the feed line coupling the common feed line to the first feed vessel comprises receiving information relating to the mass of fluid in the second feed vessel. In such examples the controller is configured to operate the first selector coupled to the common feed line so that the common feed line receives fluid from the second feed vessel instead of the first feed vessel in response to determining that the mass of fluid in the second feed vessel has reached a selected threshold.

In some examples receiving an indication that the fluid has drained from the feed line coupling the common feed line to the first feed vessel comprises at least one of (a) detecting the presence of bubbles in at least one of the common feed line, tube loop, filter and feed vessel, and (b) detecting a fall in pressure due to the presence of air, for example a drop in transmembrane pressure across a filter.

In another aspect of the disclosure there is provided a method for operating a multiple-loop tangential flow filtration apparatus, comprising:
directing fluid flow through a common feed line into a first tube loop of the multiple-loop tangential flow filtration apparatus and into a first feed vessel;
receiving information relating to the mass of fluid in the first feed vessel;
in response to determining that the mass of fluid in the first feed vessel has fallen below a selected threshold, directing fluid flow through the common feed line into a second tube loop and into the second feed vessel.

In some examples the method comprises:
opening a valve to allow air to flow into the first feed vessel to drain the first feed vessel;
in response to determining that the mass of fluid in the first feed vessel has fallen below a second selected threshold, opening a valve to allow air to flow into a feed line coupling the common feed line to the first feed vessel;
receiving an indication that the fluid has drained from the feed line coupling the common feed line to the first feed vessel; and
in response, operating a selector coupled to the common feed line so that the common feed line receives fluid from the second feed vessel instead of the first feed vessel.

In some examples, receiving an indication that the fluid has drained from the feed line coupling the common feed line to the first feed vessel comprises receiving information relating to the mass of fluid in the second feed vessel; and
operating a selector coupled to the common feed line so that the common feed line receives fluid from the second feed vessel instead of the first feed vessel is performed in response to determining that the mass of fluid in the second feed vessel has reached a selected threshold.

In some examples, receiving an indication that the fluid has drained from the feed line coupling the common feed line to the first feed vessel comprises at least one of:
detecting the presence of bubbles in at least of the common feed line, filter or feed vessel; and
detecting a fall in pressure due to the presence of air, for example a drop in transmembrane pressure across a filter.

In another aspect of the disclosure there is provided a tangential filtration apparatus for concentrating fluids. The apparatus comprises a plurality of filter lines for receiving fluid therethrough, wherein each filter line comprises a respective filter and is coupled to a corresponding feed vessel (or optionally more than one feed vessel), and a common pump for driving the fluid across each respective filter. The plurality of filter lines are coupled to the common pump via a common feed line.

In some examples the common feed line comprises a selector for selecting which filter line to direct the fluid from the common feed line towards. Each filter line may be coupled to a common waste vessel via a common waste line, wherein the common waste line is coupled to a common filtrate pump for removing the filtrate from each filter.

In some examples the apparatus further comprises a controller for controlling the flow of fluid through the apparatus, wherein the controller is configured to receive information relating to the mass of fluid in the apparatus; determine which filter line to direct fluid through based on the received information relating to the mass of fluid; and direct the fluid through a corresponding filter line based on the determination.

The controller may further be configured to receive information relating to the transmembrane pressure of fluid across each filter in the apparatus; and control operation of the common filtrate pump based on the received pressure information.

It will be understood that the tangential filtration apparatus may be an alternating tangential flow, ATF, apparatus or a tangential flow filtration apparatus.

In examples where the apparatus is an ATF, the common pump may be configured to provide a pulsatile alternating flow such that the direction of flow of fluid across each filter alternates, and may also comprise (where compatible) any of the features of the multiple-loop tangential flow filtration apparatus described above—such as the use of selectors, air inlets, the controller, mass and/or pressure sensors, the tapering of the feed vessels and so on. In such examples where the apparatus is an ATF, the apparatus may not comprise “loops” but rather fluid paths where the fluid is cycled through alternating flow.

In examples where the apparatus is a tangential flow filtration, TFF, apparatus the apparatus may comprise any of the features of the multiple-loop tangential flow filtration apparatus described above—such as the use of selectors, air inlets, the controller, mass and/or pressure sensors, the tapering of the feed vessels and so on.

In another aspect of the disclosure there is provided a computer readable non-transitory storage medium comprising a program for a computer configured to cause a processor to perform the method of the aspect described above.

### DRAWINGS

Embodiments of the disclosure will now be described, by way of example only, with reference to the accompanying drawings, in which:

FIG. 1 shows a functional block diagram of an example multiple-loop tangential flow filtration apparatus for concentrating fluids such as biological fluids;

FIG. 2 shows an example feed vessel for use with the example apparatus shown in FIG. 1;

FIG. 3 shows another example multiple-loop tangential flow filtration apparatus for concentrating fluids such as biological fluids;

FIG. 4 shows the volume reduction that was achieved using an example two loop system of embodiments of the disclosure with water alone; and

FIG. 5 shows a flow chart of an example method for operating multiple-loop tangential flow filtration apparatus, such as the apparatus shown in FIGS. 1 and 3;

FIG. 6 shows an example tangential filtration apparatus; and

FIG. 7 shows another example tangential filtration apparatus.

### SPECIFIC DESCRIPTION

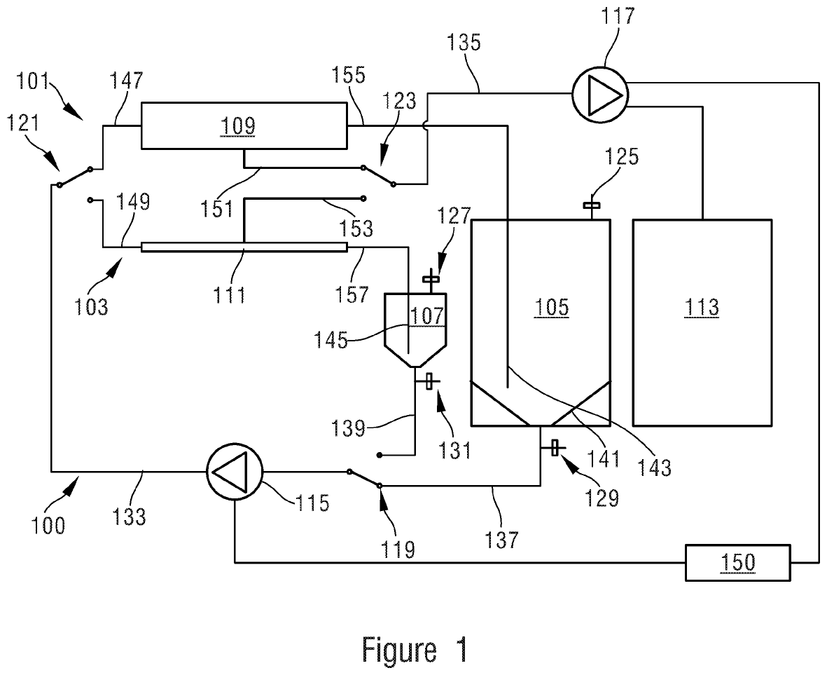

FIG. 1 shows an example multiple-loop tangential flow filtration apparatus 100 for concentrating fluids such as biological fluids. The apparatus 100 comprises a first tube loop 101 and a second tube loop 103. Each tube loop 101, 103 comprises a respective filter 109, 111, with each filter 109, 111 being coupled to two output lines.

In the example shown, a first output line 155, 157 of each filter 109, 111 is coupled to an input 143, 145 of a corresponding feed vessel 105, 107 corresponding to that tube loop 101, 103 via a corresponding output line 155, 157, such that an output line 155 from the first filter 109 of the first tube loop 101 is coupled to an input 143 of the first feed vessel 105 and an output line 157 from the second filter 111 of the second tube loop 103 is coupled to an input 145 of the second feed vessel 107. In the example shown the feed vessels 105, 107 are feed bags.

A second output line 151, 153 of each filter 109, 111 is coupled via respective waste lines 151, 153 coupled through waste selector 123 to a common waste line 135 feeding into a common waste vessel 113, which in the example shown is a common waste bag 113. A common filtrate pump 117 is coupled to the common waste line 135.

The respective feed vessels 105, 107 are coupled to a common feed line 133 via respective first and second feed lines 137, 139. A common feed pump 115 is coupled to the common feed line 133. The common feed line 133 comprises a first selector 119 at an end of the common feed line 133 proximal to the feed vessels 105, 107, and a second selector 121 at an end of the common feed line 133 proximal to the filters 109, 111. Respective filter lines 147, 149 couple the common feed line 133 to the respective filters 109, 111 via second selector 121. In the examples shown the common feed line 133 is coupled to a common feed pump 115. It will be understood that the common feed pump 115 may be configured for use with a pump head such that each tube loop 101, 103 is coupled to a common pump head via the common feed line 133. The common feed pump 115 may therefore comprise a motor, a motor control unit and a mechanical coupling for attaching to a pump head, and the pump head may comprise a fluid contacting housing with oscillating/moving parts to drive flow, and a mechanical coupling for attaching to and coupling with the corresponding mechanical coupling of the pump.

The common feed pump 115 and/or the common filtrate pump 117 may be a positive displacement pump, such as a diaphragm or gear pump, for example a Quattroflow™ pump, or a centrifugal or axial flow pump. The common feed pump 115 and/or the common filtrate pump 117 may in some examples be configured to provide a pulsatile flow. In some examples the pulsatile flow may be a pulsed alternating flow. For example, the common feed pump 115 and/or the common filtrate pump 117 may comprise a pulsed alternating flow piston or diaphragm pump.

Accordingly the first tube loop 101 comprises the first feed vessel 105, first feed line 137, the common feed line 133, selectors 119, 121, filter line 147, first filter 109 and output line 155. The second tube loop 103 comprises the second feed vessel 107, second feed line 139, common feed line 133, filter line 149, selectors 119, 121, second filter 111 and output line 157.

Also in the example shown in FIG. 1, the first tube loop 101 sits outside the second tube loop 103 such that the total path length of the first tube loop 101 is greater than the total path length of the second tube loop 103, although it will be understood that this is optional and in other examples the path length of the two tube loops 101, 103 may be the same or similar. The first tube loop 101 is configured to process a larger volume of fluid (and for example, the first tube loop 101 has a greater capacity than the second tube loop 103), and as such the first feed vessel 105 and the first filter 109 are larger than the second feed vessel 107 and the second filter 111. In the example shown, the cross-sectional area of the first filter 109 is greater than the cross-sectional area of the second filter 111, so that the first filter 109 can extract a greater volume of fluid per unit time from the first tube loop 101 than the second filter 111 can do from the second tube loop 103. In some examples the first tube loop 101 may have a larger cross-sectional internal area (such as a larger diameter) for handling a larger volume of fluid than the second tube loop 103. For example, the internal diameter of the feed line 137, filter line 147, output line 155 and optionally waste line 151 of the first tube loop 101 may be greater than the internal diameter of the feed line 139, filter line 149, output line 157 and optionally waste line 153 of the second tube loop 103. It will of course be understood that the cross-sectional internal area of the common feed line 133 will remain the same.

The common feed pump 115 is operable to draw fluid from a corresponding feed vessel 105, 107 via a corresponding feed line 137, 139 and drive the fluid through a respective tube loop 101, 103 and corresponding filter 109, 111. The first selector 119 is operable to select from which feed line 137, 139 (and therefore which feed vessel 105, 107) fluid is drawn from and into the common feed line 133. The second selector 121 is operable to select which filter line 147, 149 (and therefore to which tube loop 101, 103) fluid is drawn from the common filter line 133 and directed towards.

Each tube loop 101, 103 is therefore configured to receive fluid from the common feed line 133, filter it through a corresponding filter 109, 111 for concentration, and return the concentrated fluid (the retentate) to a corresponding feed vessel 105, 107. Accordingly in the example shown the retentate from the first tube loop 101 is returnable to the first feed vessel 105 and the retentate from the second tube loop 103 is returnable to the second feed vessel 107. In some examples the fluid (retentate) returned to a feed vessel 105, 107 may then be passed through the same tube loop 101, 103 or a different tube loop 101, 103 a number of times to increase the degree of concentration.

The common filtrate pump 117 is operable to draw waste fluid (permeate) from a corresponding tube loop 101, 103 and into the common waste vessel 113 via common waste line 135. The waste selector 123 is operable to select from which waste line 151, 153 (and therefore from which filter 109, 111 and therefore tube loop 101, 103) the common filtrate pump 117 draws the waste fluid from via common waste line 135 and into common waste vessel 113.

The apparatus is therefore configured to extract any waste (permeate) from the filters 109, 111 to the common waste vessel 113. The apparatus is configured to do this by operation of the common filtrate pump 117 to draw fluid through respective waste lines 151, 153 (selected via waste selector 123) and common waste line 135.

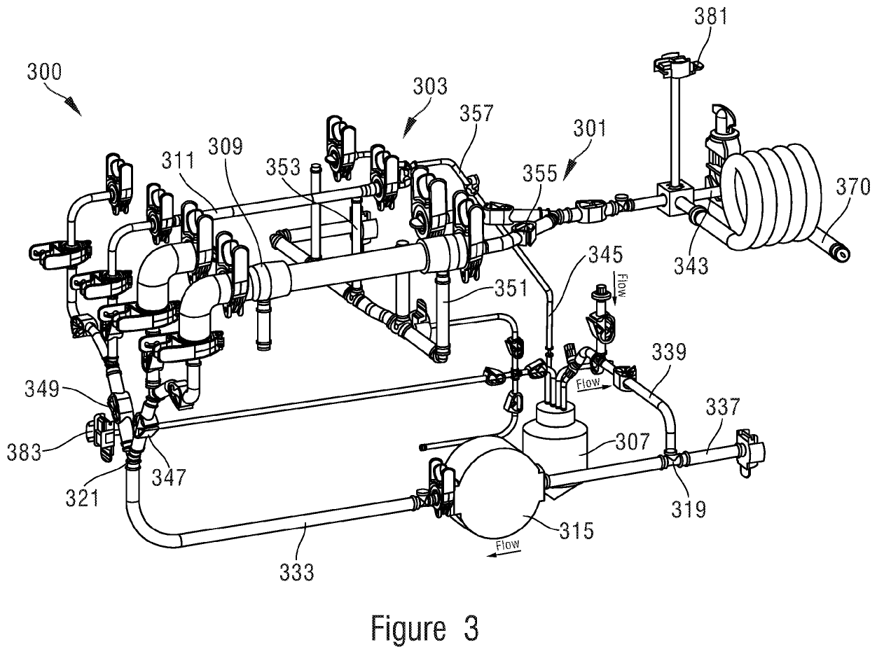

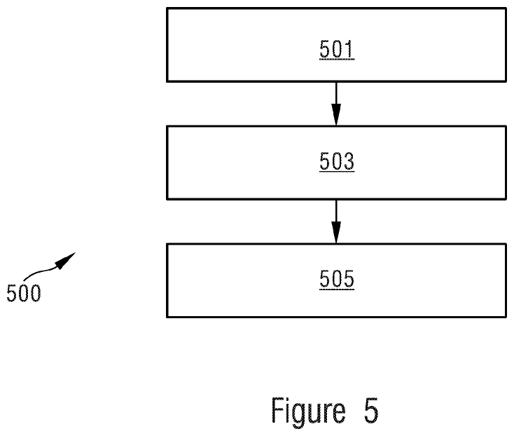

The flowchart of FIG. 5 shows an example method of operating the example apparatus shown in FIG. 1 and is also suitable for use with the apparatus shown in FIG. 3 (described in more detail below). In use, fluid is directed 501 through the common feed line 133 by common feed pump 115, into the first tube loop 101 and through filter 109, with the retentate being directed into the first feed vessel 105 and the permeate being drawn into the common waste vessel 113 by common filtrate pump 117.

The mass of fluid in at least the first feed vessel 105 (and optionally the second feed vessel 107) is monitored 503, and in response to a determination being made that the mass of fluid in the first feed vessel 105 has fallen below a selected threshold (indicating that a selected level of concentration has been reached by the first tube loop 101), fluid is directed 505 through the common feed line 133 into the second tube loop 103 and across the second filter 111, with the retentate being directed into the second feed vessel 107 (so that the second tube loop 103 can concentrate the fluid even further). Fluid is directed through the common feed line 133 into the second tube loop 103 by operating second selector 121 so that instead of fluid being directed from the common feed line 133 into first feed line 147 of the first tube loop 101, fluid is directed from the common feed line 133 into the second feed line 149 of the second tube loop 103. At a similar time, for example concurrently or subsequently, the waste selector 123 is operated so that waste (permeate) is drawn by the common filtrate pump 117 from the waste line 153 coupled to the second tube loop 103 instead of the waste line 151 coupled to the first tube loop 101.

In addition, concurrently or subsequently, the first selector 119 is operated to draw fluid from the second feed vessel 107 via second feed line 139 instead of from the first feed vessel 105 via first feed line 137. Preferably the first selector 119 is operated some time (for example a selected interval) after the second selector 121 has been operated to allow the first feed vessel 105 and optionally the first feed line 137 to drain completely, as will be described in more detail below.

In some examples there is a transition period where residual fluid in the first tube loop 101 is transferred to the second tube loop 103. During this process:
Selector 121 is switched from the first feed line 147 of the first tube loop to the second feed line 149;
The first selector 119 is connected to the first tube loop 101;
Fluid is pumped through into the second filter 111 into the second feed vessel;
The common feed pump 115 is stopped when the final material in the first feed vessel 105 enters the first feed line 137;
Residual fluid in the first feed line 137 is pumped into the common feed line 133;
The first selector 119 is then switched to connect to the second feed line 149 to feed into the second feed vessel 107 via second filter 111;
The common feed pump 115 is started again and the second tube loop 103 is primed to get rid of any air bubbles;

Once stabilised, the selector 123 is switched to start removing filtrate from the second filter 111 into the common waste vessel 113 via common filtrate pump 117.

While the apparatus shown in FIG. 1 may be operated manually, in the example shown in FIG. 1 the apparatus further comprises an optional controller 150 for controlling the flow of fluid through the apparatus 100. In the example shown in FIG. 1 the controller 150 is coupled to the common feed pump 115 and the common filtrate pump 117, although it will be understood that this is only exemplary and the controller 150 may additionally or alternatively be coupled (for example with a physical wired connection and/or wirelessly) to the selectors 119, 121, 123 and/or sensors (as will be described in more detail below). The controller 150 may be configured to receive information relating to the mass of fluid in the apparatus 100, determine which tube loop 101, 103 to direct fluid through based on the received information relating to the mass of fluid, and direct the fluid through a corresponding tube loop 101, 103 based on the determination.

For example, each feed vessel 105, 107, and optionally waste vessel 113, may be coupled to a means for determining the mass of each vessel 105, 107, 113 (for example a sensor such as a set of scales) and send sensor signals relating to the mass in each vessel 105, 107, 113 to the controller. The controller 150 may be configured to direct the fluid through a corresponding tube loop 101, 103 by operation of the first and second selectors 119, 121, and optionally common feed pump 115. In some examples the controller 150 may also be configured to control operation of the waste selector 123 and/or common filtrate pump 117 based on the determination of the mass of fluid in the apparatus 100, for example based on the determination of the mass of fluid in at least one of the first feed vessel 105, the second feed vessel 107 and the common waste vessel 113.

For example, in operation the controller 150 may be configured to identify the mass of fluid in at least the first feed vessel 105, and direct the fluid from through the first tube loop 101 to through the second tube loop 103 in response to the mass of fluid in the first feed vessel 105 falling below a selected threshold.

The controller 150 may also be configured to receive information relating to the transmembrane pressure of fluid across each filter 109, 111 of each tube loop 101, 103 in the apparatus, and control operation of the common filtrate pump 117 based on the received pressure information, for example so that the transmembrane pressure across a selected filter 109, 111 remains within a selected interval. For example, in some examples at least one of the output lines 155, 157, common waste line 135 and common feed line 133 may comprise a pressure sensor for sensing the pressure of fluid in the line. The controller 150 may be configured to receive sensor signals from at least one of these sensors and make a determination of the transmembrane pressure across at least one of the filters 109, 111 based on these received sensor signals. In other examples the controller 150 may be configured to receive sensor signals from at least one of the sensors indicating absolute pressures (i.e. with respect to atmosphere). In some examples the controller 150 may be configured to determine the transmembrane pressure based on the difference in absolute pressure values between sensors. It will be understood that in some examples there may additionally or alternatively be a single pressure sensor across at least one of the filters 109, 111 for determining the transmembrane pressure.

As noted above, in some examples the controller 150 may be configured to control operation of the common feed pump 115 and the common filtrate pump 117. The controller 150 may be configured to control operation of the common feed pump 115 and the common filtrate pump 117 based on sensor signals indicative of a pressure, for example based on a transmembrane pressure and/or based on pressure signals for examples from the common feed line 133, common waste lines 135 and/or the waste lines 151, 153. The controller may be configured to control operation of the common feed pump 115 and the common filtrate pump 117 to provide a positive and/or a negative absolute across each filter 109, 111, for example so that fluid is either forced across each filter 109, 111 and/or sucked across each filter 109, 111. In some examples the controller may also be configured to control operation of the pumps 115, 117 to reverse the flow of fluid, for example to provide a pulse alternating flow (and thereby provide alternating tangential filtration).

In some examples the apparatus 100 may also comprise one or more variable resistors coupled to respective lines, such as the waste lines 151, 153 to alter the degree of resistance each line provides to a flow and thereby to inhibit the flow of fluid through that line. The variable resistor may be configured, for example, to alter the cross-sectional internal area of the line to restrict the flow of fluid through the line. For example the variable resistor may be operable by the controller 150, and the controller 150 may be configured to alter the degree of resistance of the variable resistor to control the flow of fluid across each filter 109, 111, for example based on sensor signals indicative of a pressure such as a transmembrane pressure across a corresponding filter 109, 111.

In some examples the feed vessels 105, 107 and/or the feed lines 137, 139 may have air inlets 125, 127, 129, 131 for allowing the feed vessels 105, 107 and/or the feed lines 137, 139 to drain completely (and thus maximising the amount of concentrated sample that can be extracted from the system). For example, the first feed vessel 105 may have a first air inlet 125 for allowing the corresponding feed vessel 105 to drain completely. The first tube loop 101 may comprise a second air inlet 129 for allowing at least a portion of the first tube loop 101, for example the feed line 137, to drain completely. In such examples the controller may be configured to direct fluid to flow through the second tube 103 loop by operating the second selector 121 to direct fluid from the common feed line 133 to the filter line 149 of the second tube loop 103, and subsequently open the first air inlet 125 to allow air to flow into the first feed vessel 105 to drain the first feed vessel 105. In response to determining that the mass of fluid in the first feed vessel 105 has fallen below a second selected threshold, the controller may be configured to open the second air inlet 129 to allow air to flow into the first feed line 137 coupling the common feed line 133 to the first feed vessel 105. The controller may also be configured to receive an indication that the fluid has drained from the first feed line 137 coupling the common feed line 133 to the first feed vessel 105, and in response the controller may be configured to operate the first selector 119 coupled to the common feed line 133 so that the common feed line 133 receives fluid from the second feed vessel 107 via the second feed line 139 instead of the first feed vessel 105.

Receiving an indication that the fluid has drained from the first feed line 137 coupling the common feed line 133 to the first feed vessel 105 may comprise receiving information relating to the mass of fluid in the second feed vessel 107. For example, the controller 150 may be configured to operate the first selector 119 coupled to the common feed line 133 so that the common feed line 133 receives fluid from the second feed vessel 107 instead of the first feed vessel 105 in response to determining that the mass of fluid in the second feed vessel 107 has reached a selected threshold (for example corresponding to a mass of fluid that could be held by the first feed line 137).

Additionally or alternatively, receiving an indication that the fluid has drained from the first feed line 137 coupling the common feed line 133 to the first feed vessel 105 comprises at least one of (a) detecting the presence of bubbles in at least of the common feed line 133, a filter 109, 111 or feed vessel 105, 107, and (b) detecting a fall in pressure due to the presence of air, for example a drop in transmembrane pressure across a filter 109, 111.

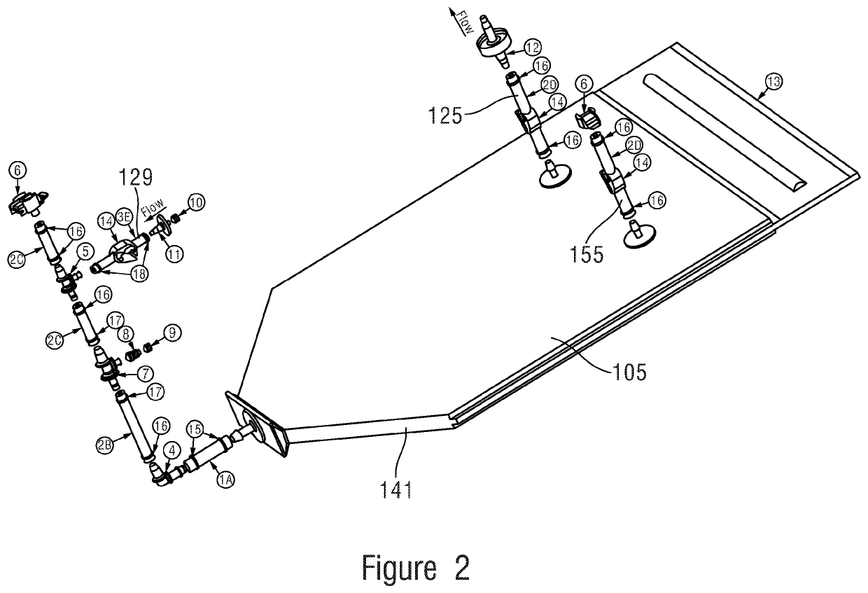

In some examples a feed vessel 105, 107 may be adapted to facilitate the draining of fluid from the feed vessel 105, 107. For example, as shown in FIG. 2 a feed vessel 105, 107 may comprise a tapered/conical portion 141 proximal to an outlet coupled to the feed line 137, 139 for delivering fluid to the common feed line 133.

In some examples the apparatus may be arranged to reduce/inhibit the likelihood of bubbles forming in the system. For example, the output line 155, 157 of a tube loop 101, 103 coupled to the input of a feed vessel 105, 107 may comprise a portion 143 that is configured to extend through the feed vessel 105, 107 proximal to the outlet coupled to the feed line 137, 139 that is in turn coupled to the common feed line 133.

In some examples the apparatus 100 may comprise one or more one-way valves to prevent the return of fluid to an undesired part of the apparatus. For example, at least one of the feed lines 137, 139, common feed line 133, filter lines 147, 149, output lines 155, 157, waste lines 151, 153 and air inlet valves 125, 127, 129 and 131 may comprise a one-way valve.

In some examples at least one of the feed vessels 105, 107 may also be coupled to a buffer vessel. The buffer vessel may be operable to increase the mass of fluid added to the feed vessel 105, 107. For example, fluid may be added from the buffer vessel to at least one of the feed vessels 105, 107 in response to the mass of fluid in that feed vessel 105, 107 falling below a selected threshold.

Although in the examples described above each tube loop 101, 103 returns fluid to a respective feed vessel 105, 107 it will be understood that in some examples a feed vessel 105, 107 may supply fluid to, and receive fluid from, a plurality of tube loops 101, 103. For example, the apparatus may only comprise the first feed vessel 105, and the output of the second tube loop 103 may also be coupled to the first feed vessel 105 such that both the first and second tube loops 101, 103 feed concentrated fluid (retentate) into the same feed vessel 105.

Although only two tube loops 101, 103 have been described above, it will of course be understood that the apparatus 100 may comprise more tube loops 101, 103. It will also be understood that each of these additional tube loops may feed fluid into a respective feed vessel and/or a common feed vessel shared with at least one other tube loop.

FIG. 3 shows another example multiple-loop tangential flow filtration apparatus 300 for concentrating fluids such as biological fluids. The apparatus 300 shown in FIG. 3 is in many respects similar to the apparatus described above with reference to FIGS. 1 and 2. In the example shown in FIG. 3, The apparatus 300 comprises a first tube loop 301 and a second tube loop 303. Each tube loop 301, 303 comprises a respective filter 309, 311, with each filter 309, 311 being coupled to two output lines.

In the example shown, a first output line 355, 357 of each filter 309, 311 is coupled to an input 343, 345 of a corresponding feed vessel corresponding to that tube loop 301, 303 via a corresponding output line 355, 357, such that an output line 355 from the first filter 309 of the first tube loop 301 is coupled to an input 343 of the first feed vessel (not shown) and an output line 357 from the second filter 311 of the second tube loop 303 is coupled to an input 345 of the second feed vessel 307.

A second output line 351, 353 of each filter 309, 311 is coupled via respective waste lines coupled through a waste selector to a common waste line (not shown) feeding into a common waste vessel (not shown). A common filtrate pump (also not shown) is coupled to the common waste line.

The respective feed vessels are coupled to a common feed line 333 via respective first and second feed lines 337, 339. A common feed pump 315 is coupled to the common feed line 333. The common feed line 333 comprises a first selector 319 at an end of the common feed line 333 proximal to the feed vessels, and a second selector 321 at an end of the common feed line 333 proximal to the filters 309, 311. In the example shown in FIG. 3, the selectors each comprise a plurality of tube clamps on respective lines operable to close off one line and open the other to select which line fluid flows from/passes to.

As with the example shown in FIG. 1, respective filter lines 347, 349 couple the common feed line 333 to the respective filters 309, 311 via second selector 321. In the examples shown the common feed line 333 is coupled to a common pump head of common feed pump 315, such that each tube loop 301, 303 is coupled to a common pump head via the common feed line 333.

The apparatus 300 shown in FIG. 3 also comprises a line 370 for coupling with a buffer vessel or respective buffer vessels, and in the example shown the line 370 for coupling with a buffer vessel is coupled to the input 343 feeding the first feed vessel. The buffer vessel may be operable to increase the mass of fluid added to each feed vessel. For example, fluid may be added from the buffer vessel to at least one of the feed vessels in response to the mass of fluid in that feed vessel falling below a selected threshold. The apparatus 300 shown in FIG. 3 also comprises lines 381, 383 coupled to the input 343, 345 of each feed vessel for coupling each feed vessel to a wash buffer for allow washing of the retentate. Wash buffer may be used once much of the media from a feed vessel has been removed, so that the wash buffer can wash the retentate prior to further filtration.

It will be understood that in examples where the apparatus 300 comprises a controller for controller the filtration process, the controller may be configured to control the delivery of fluid from a buffer vessel or a wash vessel (for example by controlling operation of a selector) based on sensor signals received, for example based on sensor signals indicative of a mass of fluid in a feed vessel. It will be understood that the controller may be coupled to the common feed pump 315 and the selectors such as selectors 319, 321.

As with the example shown in FIG. 1, the common feed pump 315 is operable to draw fluid from a corresponding feed vessel via a corresponding feed line 337, 339 and drive the fluid through a respective tube loop 301, 303 and corresponding filter 309, 311. The first selector 319 is operable to select from which feed line 337, 339 (and therefore which feed vessel) fluid is drawn from and into the common feed line 333. The second selector 321 is operable to select which filter line 347, 349 (and therefore to which tube loop 301, 303) fluid is drawn from the common filter line 333 and directed towards.

Each tube loop 301, 303 is therefore configured to receive fluid from the common feed line 333, filter it across a corresponding filter 309, 311 for concentration, and return the concentrated fluid (the retentate) to a feed vessel 305, 307.

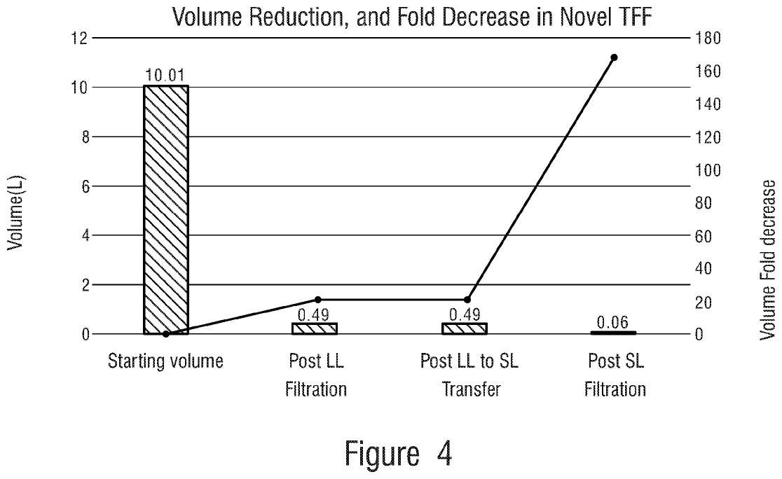

FIG. 4 shows an example volume reduction that was achieved using a two loop system as shown in FIG. 1 using water along. FIG. 4 shows a volume reduction (blue bars) and volume fold decrease (orange profile) through four stages of the process. The four stages presented are; start of the process before any filtration (Starting volume), at the end of the large loop (LL) processing (Post LL Filtration), after the material has been moved from the LL to the small loop (SL) (Post LL to SL Transfer), after the completion of the SL processing (Post SL Filtration).

Thus a significantly greater concentration of sample was achieved using the system described above with respect to FIG. 1 in a relatively short period of time (60-90 mins), as compared to currently available TFF systems. Although the system has not yet been tested with a cellular sample, it is anticipated that a similarly high level of concentration would be achieved.

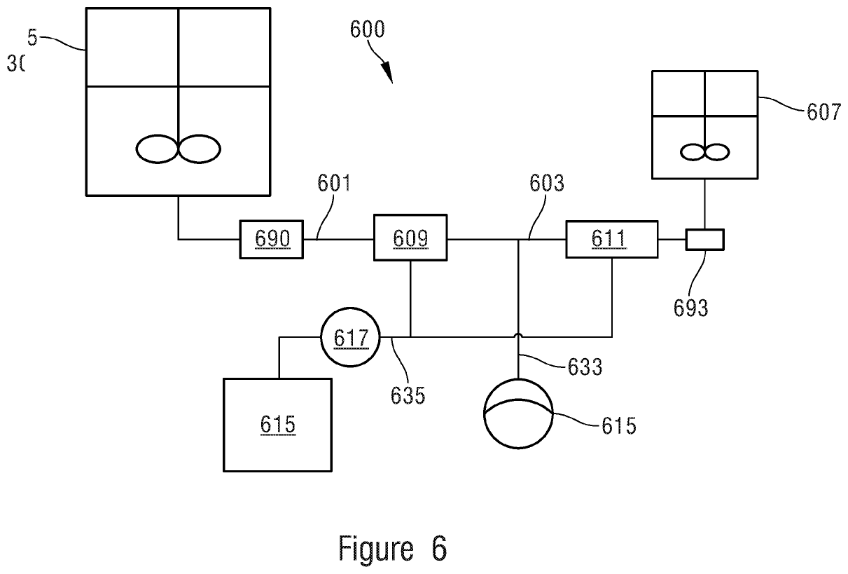

FIG. 6 shows an example tangential filtration apparatus, which in the example shown is an alternating tangential filtration, ATF, apparatus, but the general teaching of FIG. 6 may be applied to a tangential flow filtration, TFF, apparatus, and may be combined with the teaching of any of FIGS. 1 to 3 described above. In particular, any of the features of the filtration apparatus of FIGS. 1 to 3, such as the use of selectors, air inlets, the controller, mass and/or pressure sensors, the tapering of the feed vessels and so on, may be used with the apparatus of FIG. 6.

The example shown in FIG. 6 comprises a first feed vessel 605 coupled to a first filter line 601, and a second feed vessel 607 coupled to a second filter line 603. The two filter lines 601, 603 are coupled to a common feed line 633 which comprises a common pump 615. Each filter line 601, 603 comprises a respective filter 609, 611. The first feed vessel 605 is larger than the second feed vessel 607, and the corresponding filter 609 of the first filter line 601 is larger than the corresponding filter 611 of the second filter line 603. In some examples the second filter line 603 may be smaller (for example, have a smaller cross-sectional diameter) than the first filter line 601. In some examples each feed vessel 605, 607 may also comprise means for mixing the fluid in each feed vessel, such as a stirrer. The means for mixing the fluid in each vessel may be controlled by a controller (as described further below), for example based on sensor signals indicative of a mass and/or pressure of fluid in the apparatus 600.

In the example shown in FIG. 6, each filter line 601, 603 also comprises a variable resistor 690, 693 in-line with the filter 609, 611 and each feed vessel 605, 607. The variable resistors 690, 693 may be configured to act together to function as a selector to select which filter line 601, 603 to direct the fluid from the common feed line 633 towards. In the example shown in FIG. 6, each filter 609, 611 is also coupled to a common waste vessel 613 via a common waste line 635, although it will be understood that in other examples only one filter 609, 611 may be coupled to a waste vessel 613. The common waste line 635 also comprises an optional common filtrate pump 617 for removing filtrate.

In some examples the apparatus 600 further comprises a controller for controlling the flow of fluid through the apparatus. The controller may be coupled (for example with a physical wired connection and/or wirelessly) to the variable resistors 690, 693 and/or the pumps 615, 617. In some examples, as with the examples described above with respect to FIGS. 1 to 3, the apparatus 600 may further comprise sensors such as mass and/or pressure sensors and the controller may also be coupled to these sensors. As with the example apparatus described above with respect to FIGS. 1 to 3, the controller may be configured to receive information relating to the mass of fluid in the apparatus, determine which filter line to direct fluid through based on the received information relating to the mass of fluid and direct the fluid through a corresponding filter line 601, 603 (and thereby across a corresponding filter 609, 611) based on the determination. Additionally or alternatively the controller may be configured to receive information relating to the transmembrane pressure of fluid across each filter 609, 611 in the apparatus and control operation of at least one of the common pump 615 and/or the common filtrate pump 617 based on the received pressure information.

In the example shown in FIG. 6 the apparatus 600 is an alternating tangential flow, ATF, apparatus and the common pump 615 is a diaphragm pump that is configured to provide a pulsatile alternating flow such that the direction of flow of fluid across each filter 609, 611 alternates. However, it will be understood that the example apparatus 600 shown in FIG. 6 may also be used as a tangential flow filtration apparatus, TFF.

In operation, fluid flows from the first feed vessel 605 through the first filter line 601 and across the first filter 609 and into the common feed line 633. The common pump 615 is operated to provide an alternating pulsatile flow, so that fluid flows back and forth across the first filter 609. The common filtrate pump 617 may also be operated to draw filtrate from the first filter 609, and the common pump 615 and common filtrate pump 617 may be controlled by a controller based on sensor signals indicative of a mass of fluid in the first feed vessel 605 and/or a transmembrane pressure across the first filter 609. As the mass of fluid in the first feed vessel 605 decreases, the controller may for example determine that the mass of fluid in the first feed vessel 605 has reached a threshold level, and control the two variable resistors 690, 693 to instead direct fluid across the second filter 611. In this way, the fluid may be fed back and forth across the second filter 611 and into/out of the second feed vessel 607, and again the common pump 615 and common filtrate pump 617 may be controlled by a controller based on sensor signals indicative of a mass of fluid in the first feed vessel 605 and/or a transmembrane pressure across the first filter 609.

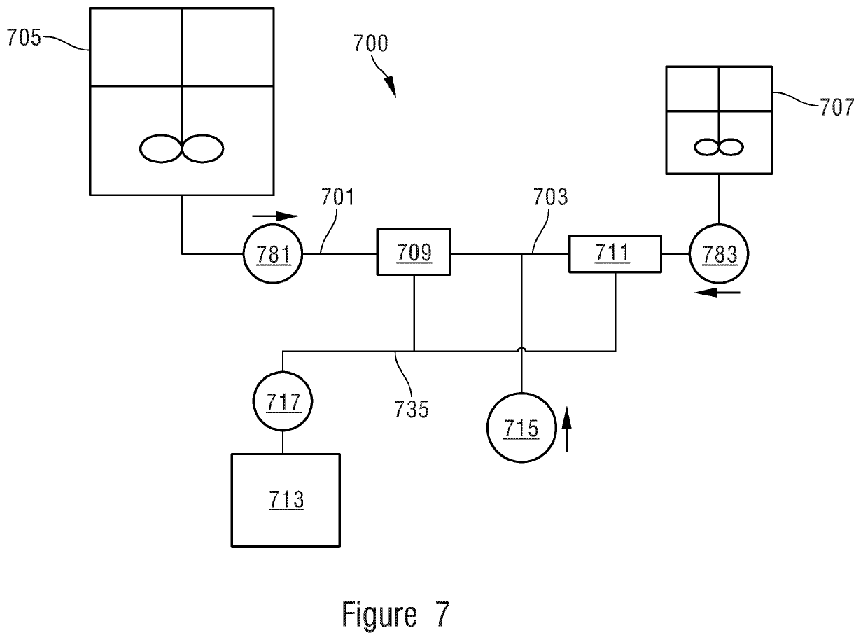

In some examples, however, it will be understood that the common pump 615 need not be an alternating pulsatile pump, and/or that there need not be two variable resistors 690, 693. For example, the apparatus shown in FIG. 7 is in many respects similar to the apparatus shown in FIG. 6 (where like reference numbers denote the same or similar features) but instead the two variable resistors 690, 693 are replaced by two respective one-way pumps 781, 783, and the common pump 715 is also a one-way pump. In such examples the controller may be configured to control operation of the one-way pumps 781, 783, 715 to provide an alternating flow across each filter 709, 711. For example, the controller may be configured to control operation of the one way pump 781 on the first filter line 701 in concert with the common pump 715 to provide an alternating flow across the first filter 609, and then control operation of the one way pump 783 on the second filter line 703 in concert with the common pump 715 to provide an alternating flow across the second filter 611.

It will be understood that in the context of the present disclosure the use of the word line refers to a tube or pipe capable of transporting a fluid.

It will also be understood that in the context of the present disclosure, although reference is made to feed and waste vessels, that any other vessel suitable for holding fluid such as liquid may be used, such as a bag or container.

It will also be understood that in the context of the present disclosure the term fluid may encompass a fluid comprising biological material, e.g. comprising cellular material (such as lymphocytes, e.g. T cells or NK cells) or virus (including viral vectors), e.g. AAV, lentivirus or gammaretrovirus. The fluid may of course be a liquid.

It will also be appreciated from the discussion above that the embodiments shown in the Figures are merely exemplary, and include features which may be generalised, removed or replaced as described herein and as set out in the claims.

In the context of the present disclosure other examples and variations of the apparatus and methods described herein will be apparent to a person of skill in the art.

1. A multiple-loop tangential flow filtration apparatus configured to concentrate fluids, the apparatus comprising:
a plurality of tube loops for receiving fluid therethrough, each tube loop comprising a respective filter; and
a common feed pump for driving the fluid across each respective filter;
wherein the plurality of tube loops are coupled to the common feed pump via a common feed line.

2.-32. (canceled)

33. The multiple-loop tangential flow filtration apparatus of claim 1, wherein each tube loop is coupled to a respective feed vessel and the apparatus is configured so that each tube loop supplies retentate from each tube loop to a corresponding respective feed vessel.

34. The multiple-loop tangential flow filtration apparatus of claim 1, further comprising a first tube loop coupled to a corresponding first feed vessel, and a second tube loop coupled to a corresponding second feed vessel,
wherein an input of each tube loop is coupled to the common feed line, and an output of the first tube loop is coupled to an input of the first feed vessel and an output of the second tube loop is coupled to an input of the second feed vessel such that the retentate from the first tube loop is returnable to the first feed vessel and the retentate from the second tube loop is returnable to the second feed vessel.

35. The multiple-loop tangential flow filtration apparatus of claim 34, wherein the common feed line comprises a selector configured to select which feed vessel from which to draw fluid into the common feed line.

36. The multiple-loop tangential flow filtration apparatus of claim 1 wherein:
each of the plurality of tube loops is configured to process a different flow rate of fluid,
wherein each of the plurality of tube loops has a different internal cross-sectional area, and
wherein tube loops with a smaller internal cross-sectional area have a shorter total loop path length than tube loops with a larger internal cross-sectional area.

37. The multiple-loop tangential flow filtration apparatus of claim 1, wherein each tube loop is coupled to a common waste vessel via a common waste line, and wherein the common waste line is coupled to a common filtrate pump configured to remove the filtrate from each filter.

38. The multiple-loop tangential flow filtration apparatus of claim 37, wherein the common waste line comprises a switch configured to select which tube loop filtrate is received from.

39. The multiple-loop tangential flow filtration apparatus of claim 1, further comprising a controller configured to control the flow of fluid through the apparatus, wherein the controller is configured to:
receive information relating to the mass of fluid in the apparatus;
determine which tube loop to direct fluid through based on the received information relating to the mass of fluid; and
direct the fluid through a corresponding tube loop based on the determination.

40. The multiple-loop tangential flow filtration apparatus of claim 1, wherein:
each tube loop is coupled to a common waste vessel via a common waste line,
the common waste line is coupled to a common filtrate pump configured to remove the filtrate from each filter, further comprising a controller configured to control the flow of fluid through the apparatus, and
the controller is configured to:
receive information relating to the mass of fluid in the apparatus;
determine which tube loop to direct fluid through based on the received information relating to the mass of fluid;
direct the fluid through a corresponding tube loop based on the determination;
receive information relating to the pressure in each tube loop, such as the transmembrane pressure of fluid across each filter of each tube loop in the apparatus; and
control operation of the common filtrate pump based on the received pressure information.

41. A multiple-loop tangential flow filtration apparatus configured to concentrate fluids, the apparatus comprising:
a first tube loop comprising a corresponding first feed vessel and a first filter;
a second tube loop comprising a corresponding second feed vessel and a second filter;
a common pump for driving the fluid across each respective filter of each tube loop, wherein the first and second tube loops are coupled to the common pump via a common feed line;
wherein the common feed line comprises:
a first selector configured to select which feed vessel from which to draw fluid into the common feed line; and
a second selector configured to select which filter of each tube loop to direct the fluid from the common feed line towards; and
a controller configured to control the flow of fluid through the apparatus, wherein the controller is configured to:
receive information relating to the mass of fluid in at least one of the feed vessels;
determine which tube loop to direct fluid through based on the received information relating to the mass of fluid in at least one of the feed vessels; and
direct the fluid through a corresponding tube loop based on the determination by controlling operation of the first and second selectors.

42. The multiple-loop tangential flow filtration apparatus of claim 41, wherein the controller is configured to determine to direct fluid through the second tube loop in response to the mass of fluid in the first feed vessel falling below a selected threshold.

43. The multiple-loop tangential flow filtration apparatus of claim 42, wherein:
the first feed vessel has a first air inlet configured to allow the first feed vessel to drain completely and the first tube loop comprises a second air inlet configured to allow at least a portion of the first tube loop to drain completely; and
wherein the controller is configured to direct fluid to flow through the second tube loop, wherein the controller is configured to:
operate the second selector to direct fluid from the common feed line the second tube loop;
open the first air inlet to allow air to flow into the first feed vessel to drain the first feed vessel;
in response to a determination that the mass of fluid in the first feed vessel has fallen below a second selected threshold, open the second air inlet to allow air to flow into a feed line coupling the common feed line to the first feed vessel;
receive an indication that the fluid has drained from the feed line coupling the common feed line to the first feed vessel; and
in response, operate the first selector coupled to the common feed line so that the common feed line receives fluid from the second feed vessel instead of the first feed vessel.

44. The multiple-loop tangential flow filtration apparatus of claim 43, wherein receipt of an indication that the fluid has drained from the feed line coupling the common feed line to the first feed vessel comprises receipt of information relating to the mass of fluid in the second feed vessel; and
wherein the controller is configured to operate the first selector coupled to the common feed line so that the common feed line receives fluid from the second feed vessel instead of the first feed vessel in response to a determination that the mass of fluid in the second feed vessel has reached a selected threshold.

45. The multiple-loop tangential flow filtration apparatus of claim 43, wherein receipt of an indication that the fluid has drained from the feed line coupling the common feed line to the first feed vessel comprises at least one of:
a detection of a presence of bubbles in at least one of the common feed line, filter or feed vessel; and
a detection of a fall in pressure due to the presence of air related to a drop in transmembrane pressure across a filter.

46. A tangential filtration apparatus configured to concentrate fluids, the apparatus comprising:
a plurality of filter lines configured to receive fluid therethrough, wherein each filter line comprises a respective filter and is coupled to a corresponding feed vessel; and
a common pump configured to drive the fluid across each respective filter;
wherein the plurality of filter lines is coupled to the common pump via a common feed line.

47. The tangential filtration apparatus of claim 46, further comprising at least one selector configured to select which filter line to direct the fluid from the common feed line towards.

48. The tangential filtration apparatus of claim 46, wherein each filter line is coupled to a common waste vessel via a common waste line, and wherein the common waste line is coupled to a common filtrate pump configured to remove the filtrate from each filter.

49. The tangential filtration apparatus of claim 46, further comprising a controller configured to control the flow of fluid through the apparatus, wherein the controller is configured to:
receive information relating to the mass of fluid in the apparatus;
determine which filter line to direct fluid through based on the received information relating to the mass of fluid; and
direct the fluid through a corresponding filter line based on the determination.

50. The tangential filtration apparatus of claim 46, wherein the apparatus is an alternating tangential flow (ATF) apparatus and the common pump is configured to provide a pulsatile alternating flow such that the direction of flow of fluid across each filter alternates.

51. The tangential filtration apparatus of claim 46, wherein the apparatus is a tangential flow filtration (TFF) apparatus.
